SEQUENCE LISTING (16 sequences)

SEQ ID NO: 1 - DNA, 19 bp, Homo sapiens
cccaggcattgctgacagg

SEQ ID NO: 2 - DNA, 20 bp, Homo sapiens
tggaaggtggacagtgaggc

SEQ ID NO: 3 - DNA, 21 bp, Homo sapiens
cccgggacttcatcaatcagt

SEQ ID NO: 4 - DNA, 18 bp, Homo sapiens
tcccggagctggtaggtg

SEQ ID NO: 5 - DNA, 21 bp, Homo sapiens
cagctttgcatgatggcgtct

SEQ ID NO: 6 - DNA, 20 bp, Homo sapiens
gaagggtacaggcgagttgg

SEQ ID NO: 7 - DNA, 19 bp, Homo sapiens
gcgcagcactcttccatcc

SEQ ID NO: 8 - DNA, 20 bp, Homo sapiens
tcgtcgcgtccttggtttta

SEQ ID NO: 9 - DNA, 20 bp, Homo sapiens
cttgcctaatccacagactg

SEQ ID NO: 10 - DNA, 18 bp, Homo sapiens
gcctgaacagcaccacta

SEQ ID NO: 11 - DNA, 20 bp, Homo sapiens
tctcccaggaatacaacgat

SEQ ID NO: 12 - DNA, 20 bp, Homo sapiens
acaggtcattgtcacagcac

SEQ ID NO: 13 - DNA, 20 bp, Homo sapiens
caggcttgctgaacgtgaag

SEQ ID NO: 14 - DNA, 18 bp, Homo sapiens
ggagcaccttggcgaaca

SEQ ID NO: 15 - DNA, 19 bp, Homo sapiens
ccgggccacgtagaaaaca

SEQ ID NO: 16 - DNA, 20 bp, Homo sapiens
cctccaaacacagccaggac

(19) United States
(12) Patent Application Publication
(10) Pub. No.: US 2011/0054033 A1
(43) Pub. Date: Mar. 3, 2011

(54) METHODS OF USING ABSCISIC ACID FOR AMELIORATING HYPERTENSION AND VASCULAR INFLAMMATION

(75) Inventors: Josep Bassaganya-Riera, Blacksburg, VA (US); Raquel Hontecillas, Blacksburg, VA (US); Amir Guri, San Diego, CA (US)

(73) Assignee: Virginia Tech Intellectual Properties, Inc., Blacksburg, VA (US)

(21) Appl. No.: 12/869,237

(22) Filed: Aug. 26, 2010

Related U.S. Application Data
(60) Provisional application No. 61/236,905, filed on Aug. 26, 2009.

(51) Int. Cl. A61K 31/192; A61P 9/12

(52) U.S. Cl. 514/570

(57) ABSTRACT

Methods and compositions for treating or preventing hypertension or vascular inflammation are described. These methods of the invention involve the administration of abscisic acid (ABA) to subjects in need thereof.

This application claims the priority of U.S. Provisional Patent Application No. 61/236,905, filed Aug. 26, 2009, the disclosure of which is incorporated herein by reference.

### FIELD OF THE INVENTION

The present invention relates to the use of a therapeutically or prophylactically effective amount of abscisic acid to prevent or treat hypertension or vascular inflammation, particularly atherosclerosis.

### BACKGROUND OF THE INVENTION

In spite of efforts by public health officials to encourage physical activity and reduce energy intake, the obesity rate in the U.S. and worldwide has continued to climb and it has reached epidemic proportions. According to estimates by the Center for Disease Control and Prevention in the year 2000, 30% of Americans are obese and 65% are overweight (CDC: National Diabetes Fact Sheet: general information and national estimates on diabetes in the United States, 2005. In U.S. Department of Health and Human Services, Center for Disease Control and Prevention, 2005 Atlanta, Ga., 2005, p. 1-10). One of the manifestations associated with this obesity epidemic is cardiovascular disease (CVD). CVD represents a major health risk throughout the industrialized world. Atherosclerosis, the most prevalent of cardiovascular diseases, is the principal cause of heart attack, stroke, and gangrene of the extremities. It is also the principal cause of death in the United States.

Atherosclerosis is a complex disease involving many cell types and molecular factors. The process is believed to occur as a response to insults to the endothelial cell layer that lines the wall of the artery. The process includes the formation of fibrofatty and fibrous lesions or plaques, preceded and accompanied by inflammation. The advanced lesions of atherosclerosis may occlude an artery, and result from an excessive inflammatory-fibroproliferative response to numerous different forms of insult. For example, shear stresses are thought to be responsible for the frequent occurrence of atherosclerotic plaques in regions of the circulatory system where turbulent blood flow occurs, such as branch points and irregular structures.

The first event that is observed in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDL's are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

The lipid-filled monocytes are termed “foam cells,” and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque. Such plaques occlude the blood vessel concerned and restrict the flow of blood, resulting in ischemia which is characterized by a lack of oxygen supply in tissues of organs due to inadequate perfusion. The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented.

Hypertension, which affected around 26.4% of the global adult population in 2000 and was projected to affect 29.2% by 2025 (Kearney et al., Lancet 365:217-223, 2005), is the major controllable risk factor associated with cardiovascular disease (CVD) events such as myocardial infraction, stroke, heart failure, and end-stage diabetes. A 5-mmHg reduction in blood pressure has been equated with around 16% reduction in CVD (FitzGerald et al., J. Nutr. 134: 980S-988S, 2004). The seventh Joint National Committee (JNC 7) reported the risk of heart disease and stroke increases at blood pressure above systolic blood pressure (SBP)/diastolic blood pressure (DBP) values of 115/75 mmHg. JNC 7 recommended that the pre-hypertensive individuals (SBP 120-139 mmHg or DBP 80-89 mmHg) adopt health-promoting lifestyle modifications to prevent the progressive rise in blood pressure and CVD. Therefore, the awareness of and demand for functional food ingredients or nutraceuticals for controlling CVD, particularly hypertension and vascular inflammation, have been raised globally.

### SUMMARY OF THE INVENTION

It is an object of the present invention to provide methods for the treating or preventing hypertension by administering to a mammal a composition containing abscisic acid (ABA). The amount of ABA is sufficient to alter the expression or activity of eNOS and/or 15-LOX in a cell of the mammal. Preferably, the amount of ABA is sufficient to increase expression or activity of endothelial nitric oxide synthase (eNOS) and/or 15-lipoxygenase (15-LOX) in the aortic wall, or to increase nitric oxide (NO) release in the endothelial cells of the mammal.

It is a further object of the present invention to provide methods for the treating or preventing vascular inflammation in a mammal by administering to the mammal a composition containing ABA. The amount of ABA is sufficient to alter the expression or activity of monocyte chemotactic protein-1 (MCP-1) and/or vascular cell adhesion molecule-1 (VCAM-1). Preferably, the amount of ABA is sufficient to decrease the expression or activity of MCP-1 or VCAM-1. Alternatively, the amount of ABA is sufficient to prevent the infiltration of macrophages and lymphocytes into atherosclerotic plaques of the mammal. The vascular inflammation includes, but is not limited to, atherosclerosis, myocardial infarction, vasculitis, and stroke. Preferably, the vascular inflammation is atherosclerosis.

It is yet a further object of the present invention to provide methods for the treating or preventing atherosclerosis in a mammal by administering to the mammal a composition containing ABA. The amount of ABA is sufficient to alter the expression or activity of MCP-1 and/or VCAM-1. Preferably, the amount of ABA is sufficient to decrease the expression or activity of MCP-1 or VCAM-1.

It is yet another object of the present invention to provide methods for preventing, in a mammal, the infiltration of macrophages and lymphocytes into atherosclerotic plaques or blood vessels with inflammatory lesions by administering to the mammal a composition containing ABA. The amount of ABA is sufficient to alter the expression or activity of MCP-1 and/or VCAM-1. Preferably, the amount of ABA is sufficient to decrease the expression or activity of MCP-1 or VCAM-1 in the mammal.

The compositions of the present invention contain ABA and, optionally, a carrier. Preferably, that carrier has substantially no effect on eNOS, 15-lipoxygenase, NO release, MCP-1, VCAM-1, or the infiltration of macrophages and lymphocytes into artherosclerotic plaques. The carriers of the present invention may be pharmaceutical carriers, nutritional supplements, functional foods, or dietary aids, as are further described below.

### BRIEF DESCRIPTION OF THE DRAWINGS

FIG. 1 is a diagram showing the chemical formulas for the general structure for (A) ABA; and (B) a structurally related compound.

FIG. 2 is a graph showing fast performance liquid chromatography (FPLC) separation of plasma lipoproteins performed on a Superose 6 column using buffer containing 0.15M NaCl, 0.01M Na₂HPO₄ and 1 mM EDTA at a flow rate of 0.5 ml/min. Forty 0.5 ml fractions were collected and cholesterol was measured in fractions 10-40. Fractions 15-20 contain VLDL, fractions 21-27 contain LDL and fractions 28-34 contain HDL.

FIG. 3 is a graph showing that ABA decreases systolic blood pressure in ApoE (−/−) mice. ApoE −/− mice were fed high fat diets created with (solid line) or without (dashed line) 100 mg/kg diet racemic abscisic acid (ABA). Systolic blood pressure was assessed on days 0, 28, 56, and 72 of dietary treatment. Data were analyzed as a completely randomized design. Data points with an asterisk are significantly different (P<0.05).

FIG. 4 are photos and graphs showing aortas collected on day 84 of dietary intervention. Representative photomicrographs (100×) of aortic root specimens from (A) control and (B) ABA-fed mice. (C) Analysis of lumenal areas and (D) tunica media areas were performed with aortic cross-sections using Image Pro Plus software. (E) Aortic thickness was analyzed as a ratio of the intima to the media. Gene expression in the aorta of (F) endothelial nitric oxide synthase (eNOS) and (G) 15-lipoxygenase (15-LOX) were measured as a ratio to the house-keeping gene β-actin. Data were analyzed as a completely randomized design. Data points with an asterisk are significantly different (P<0.05).

FIG. 5 are photos and graphs showing histological examination of aortic lesions. ApoE −/− mice were fed high fat diets created with or without abscisic acid (ABA, 100 mg/kg diet). After 84 days aortas were excised and lesions were analyzed histologically according to methods described by van Vlijmen et al[13]. Representative photomicrographs (100×) of a (A) mild and (B) severe lesion are depicted. (C) The total number of mild (categories 1-3) and severe (categories 4-5) lesions from control and ABA-fed mice are shown. (D) Lesional areas, as a percent of total lumen area, for control and ABA-fed mice were calculated from histological sections with Image Pro Plus software. Data are represented as mean±standard error. Points with an asterisk are significantly different from the control (P<0.05).

FIG. 6 are graphs showing that ABA decreases immune cell infiltration in aortas of ApoE−/− mice and reduces pro-inflammatory gene expression. ApoE −/− mice were fed high fat diets created with or without 100 mg/kg diet racemic abscisic acid (ABA). (A) Infiltration of F4/80⁺CD11b⁺ macrophages and CD4⁺ T-cells into the aorta were assessed with flow cytometry. Gene expression in the aorta of pro-inflammatory markers (B) vascular cell adhesion molecule 1 (VCAM-1), (C) monocyte chemoattractant protein 1 (MCP-1), (D) E-selectin, (F) peroxisome proliferator-activated receptor γ (PPAR γ), and (G) CD36 were measured as a ratio to the housekeeping gene β-actin. (H) MCP-1 protein concentrations (pg/mL) were measured by ELISA. Data were analyzed as a completely randomized design. Data points with an asterisk are significantly different (P<0.05).

FIG. 7 are graphs showing effect of ABA on (A) cAMP; (B) NO; and (C) eNOS. (A) Confluent human aortic endothelial cell (HAEC) were serum-starved using HBSS buffer for 30 min and then stimulated with various concentrations of ABA (0, 0.01, 0.1, 1, 10 μM) or Forskolin (1 μM) for 5 minutes. After removing supernatant, cells were lysated with 0.1 M HCL for 20 minutes to measure non-acetylated cAMP. (B) Confluent HAEC were serum-starved using HBSS buffer for 30 minutes and then stimulated with various concentrations of ABA (0, 0.001, 0.01, 0.1, 1, 10 μM) for 15 minutes to measure NO. Results (pmol/ml) from four independent experiments are depicted. (C) Endothelial nitric oxide synthase (eNOS) mRNA expression after 24 hr treatment with control vehicle (DMSO) or ABA (10 μM). Data are represented as mean±standard error. Points with an asterisk are significantly different from the control (P<0.05).

### DETAILED DESCRIPTION OF THE PREFERRED EMBODIMENTS

The present invention provides new uses for abscisic acid and structurally related compounds. The term abscisic acid (abbreviated herein as ABA) herein refers to a plant hormone containing a trimethylcyclohexene ring with one or more hydroxy groups (for instance a 6-hydroxy group), a 3-oxo group and an unsaturated side chain in the sixth position of the trimethylcyclohexene ring containing cis-7, trans-9 double bonds, its non-toxic salts, active esters, active isomers, active metabolites, and mixtures thereof. Non-toxic salts include, for example, alkyl esters having from 1 to 6 carbon atoms in the alkyl group, as well as mono-, di- and tri-glycerides, and mixtures thereof. Active isomers of abscisic acid include geometrical isomers and its non-toxic salts, e.g., sodium, potassium, calcium, and magnesium salts, and its active esters, e.g., alkyl esters having from 1 to 6 carbon atoms in the alkyl group, as well as mono-, di- and tri-glycerides, and mixtures thereof. Active optical isomers of abscisic acid include the (+)-enantiomer and the (−)-enantiomer and its non-toxic salts, e.g., sodium, potassium, calcium, and magnesium salts, and its active esters, e.g., alkyl esters having from 1 to 6 carbon atoms in the alkyl group, as well as mono-, di- and tri-glycerides, and mixtures thereof. Active metabolites of abscisic acid include oxygenated abscisic acid analogs, including but not limited to, 8′-hydroxyABA, (+)-7′-hydroxyABA, 2′3′-dihydroABA, 8′-hydroxy-2′,3′-dihydroABA and its non-toxic salts, e.g., sodium, potassium, calcium, and magnesium salts, and its active esters, e.g., alkyl esters having from 1 to 6 carbon atoms in the alkyl group, as well as mono-, di- and tri-glycerides, and mixtures thereof. Structurally related compounds, include but are not limited to, compounds containing conjugated double bonds (e.g., conjugated dienes, trienes and tetraenes) in the unsaturated side chain and compounds containing a trimethylcyclohexene ring with or without hydroxy moieties. For ease of reference, all such compounds are referred to herein generally at times as abscisic acid or ABA.

The ABA may be a substantially pure single chemical compound or a mixture of one or more ABA compounds as defined above. For example, the ABA may be in the form of an extract obtainable or obtained from plant extracts, either directly or following one or more steps of purification or it can be chemically synthesized.

ABA has previously been extracted from leaves of Lupin (Lupinus cosentinii), Apricot (Prunus armeniaca), Avocado (Persea Americana), Sunflower (Helianthus annuus), Grapevine (Vitis vinifera), Tomato (Lycopersicon esculentum), Spinach (Spinacia oleracea), Orange (Citrus sinensis) and Mango (Mangifera indica) (Loveys et al., Aust. J. Plant Physiol. 15: 421-427, 1988, which is incorporated herein by reference). ABA and its metabolites have also been isolated from Brassica napus and Brassica rapa seed (Zhou et al., Plant Physiol 134: 361-369, 2004, which is incorporated herein by reference) and could also be isolated from fruits and any other plant materials. The ABA compound has been extracted from plant leaves through many procedures, including: 1) methanol extraction; 2) cold water extraction or 3) boiling water extraction (Loveys et al. 1988). For the methanol extraction, samples of leaf material were homogenized in aqueous methanol, the homogenate was centrifuged and the pellet re-extracted with methanol. Water was added to the combined supernatants before evaporation. The resulting extract was adjusted to a pH of 2.5 and the abscisic acid compound extracted with three washes of ethyl acetate. The ethyl acetate extracts can be further purified by chromatography. The cold water and boiling water methods consist of homogenization of plant materials in cold or boiling water, respectively prior to the ethyl acetate extraction.

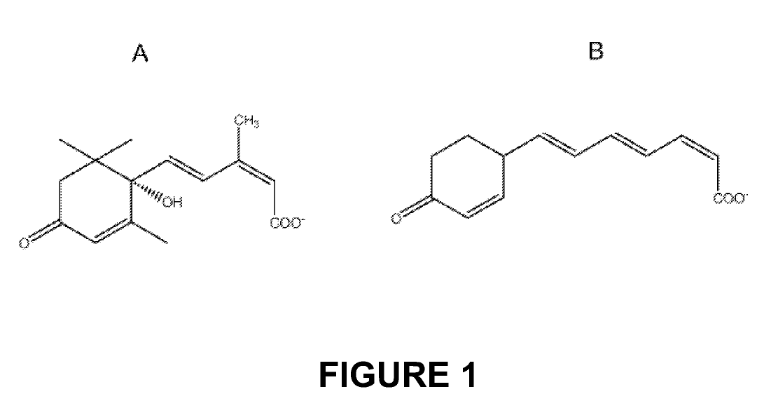

The ABA used in the described methods may be in a free acid form or bound chemically through ester linkages. In its natural form, ABA is heat stable. ABA may be used in its natural state or in a dried and powdered form. Further, the free acid form of ABA may be converted into a non-toxic salt, such as sodium, potassium or calcium salts, by reacting chemically equivalent amounts of the free acid form with an alkali hydroxide at a basic pH. FIG. 1 depicts ABA and an exemplary compound falling within the definition of ABA and structurally related compounds. Other structurally related compounds are known in the art, such as those disclosed by Hill et al. (Plant Physiol. 108:573-57, 1995), which is hereby incorporated herein by reference. As used throughout this document, the term ABA and all of its forms are meant to include the following compounds: abscisic acid, esters thereof, pharmaceutically suitable salts thereof, metabolites thereof, structurally related compounds thereof, analogs thereof, or combinations thereof, as disclosed herein.

In general, the invention provides for the use of ABA (and structurally related compounds, such as a compound selected from the group consisting ABA, esters thereof, pharmaceutically suitable salts thereof, metabolites thereof, structurally related compounds thereof, or combinations thereof) in the treatment and prevention of hypertension and vascular inflammation. The invention is based, at least in part, on the discovery that ABA can ameliorate vascular inflammation (and thus atherosclerosis) and hypertension, possibly by different mechanisms. Those effects result from exposing cells to ABA. In certain embodiments, the invention provides for treating a subject with ABA, for example as a dietary supplement. It also provides for treating a subject suffering from hypertension or vascular inflammation.

In one embodiment, the invention provides a method of treating a subject suffering from or at risk of suffering from hypertension. In general, the method comprises administering ABA or a composition comprising ABA to a subject in need thereof. The amount of ABA is sufficient to alter the expression or activity of eNOS and/or 15-LOX in a cell of the mammal. Preferably, the amount of ABA is sufficient to increase expression or activity of endothelial nitric oxide synthase (eNOS) and/or 15-lipoxygenase (15-LOX) in the aortic wall, or to increase nitric oxide (NO) release in the endothelial cells of the mammal. The ABA may affect the expression of the eNOS or 15-LOX gene, resulting in a change in eNOS or 15-LOX mRNA levels in a cell. The ABA may affect the amount of the eNOS or 15-LOX protein in a cell, preferably by increasing the expression of eNOS or 15-LOX gene. The ABA may also affect the activity of the eNOS or 15-LOX protein in a cell, preferably by increasing the amount of eNOS or 15-LOX in the cell. In general, the method comprises administering a sufficient amount for a sufficient time to see a change in eNOS or 15-LOX expression or activity, or change in NO release. Often, the amount administered and the amount of time is adequate to see a change in one or more clinical symptoms of hypertension, or to stop progression of hypertension from reaching a stage where one or more clinical symptoms are seen. According to this aspect, ABA and its related compounds can be used to treat a subject therapeutically or prophylactically, and to prepare a composition for use in treating hypertension.

In another embodiment, the invention provides a method of treating a subject suffering from or at risk of suffering from vascular inflammation (and thereby ameliorating atherosclerosis). In general, the method comprises administering ABA or a composition comprising ABA to a subject in need thereof. The amount of ABA is sufficient to alter the expression or activity of MCP-1 and/or VCAM-1. Preferably, the amount is sufficient to decrease the expression or activity of MCP-1 or VCAM-1 in the subject. The ABA may affect the expression of the MCP-1 or VCAM-1 gene, resulting in a change in MCP-1 or VCAM-1 mRNA levels in a cell. The ABA may affect the amount of the MCP-1 or VCAM-1 protein in a cell, preferably by increasing the expression of MCP-1 or VCAM-1 gene. The ABA may also affect the activity of the MCP-1 or VCAM-1 protein, preferably by increasing the amount of MCP-1 or VCAM-1. In general, the method comprises administering a sufficient amount for a sufficient time to see a change in MCP-1 or VCAM-1 expression or activity. Often, the amount administered and the amount of time is adequate to see a change in one or more clinical symptoms of hypertension, or to stop progression of hypertension from reaching a stage where one or more clinical symptoms are seen. According to this aspect, ABA and its related compounds can be used to treat a subject therapeutically or prophylactically, and to prepare a composition for use in treating vascular inflammation or atherosclerosis.

In yet another embodiment, the invention provides a method for preventing the infiltration of macrophages and lymphocytes into an atherosclerotic plaque of a subject. In general, the method comprises administering ABA or a composition comprising ABA to the subject, particular those suffering from atherosclerosis. The amounts of ABA used are similar to the method for treating vascular inflammation described above.

According to the invention, the term “a subject suffering from [a disease or condition]” is used to mean a subject (e.g., animal, human), preferably a mammal, having the disease or condition showing one or more clinical signs that are typical of that disease or condition. The term “a subject at risk for developing [a disease or condition]” is used to mean a subject in which one or more clinical signs of the disease or condition are not clearly shown, but who shows one or more sub-clinical signs that are typical of the disease or condition, or who has a family history that indicates a significant risk of developing the disease or condition, or who suffers from symptoms that may develop into the disease or condition. In general, the method of treating or preventing according to the invention comprises administering to the subject an amount of ABA that is effective in treating or preventing one or more symptoms or clinical manifestations of the disease, or in preventing development of such symptom(s) or manifestation(s).

The terms “preventing” or “treating” or “ameliorating” and similar terms used herein, include prophylaxis and full or partial treatment. The terms may also include reducing symptoms, ameliorating symptoms, reducing the severity of symptoms, reducing the incidence of the disease, or any other change in the condition of the patient, which improves the therapeutic outcome.

In the methods, administration of ABA can be through any known and acceptable route. Such routes include, but are not necessarily limited to, oral, via a mucosal membrane (e.g., nasally, via inhalation, rectally, intrauterally or intravaginally, sublingually), intravenously (e.g., intravenous bolus injection, intravenous infusion), intraperitoneally, and subcutaneously. Administering can likewise be by direct injection to a site (e.g., organ, tissue) containing a target cell (i.e., a cell to be treated). Furthermore, administering can follow any number of regimens. It thus can comprise a single dose or dosing of ABA, or multiple doses or dosings over a period of time. Accordingly, treatment can comprise repeating the administering step one or more times until a desired result is achieved. In embodiments, treating can continue for extended periods of time, such as weeks, months, or years. Those of skill in the art are fully capable of easily developing suitable dosing regimens for individuals based on known parameters in the art. The methods thus contemplate controlling, but not necessarily eliminating, the disease or disorder.

The amount to be administered will vary depending on the subject, stage of disease or disorder, age of the subject, general health of the subject, and various other parameters known and routinely taken into consideration by those of skill in the medical arts. As a general matter, a sufficient amount of ABA will be administered in order to make a detectable change in the amount or activity of eNOS, 15-LOX, MCP-1, or VCAM-1; or change in the NO release from endothelial cells. Suitable amounts are disclosed herein, and additional suitable amounts can be identified by those of skill in the art without undue or excessive experimentation, based on the amounts disclosed herein.

The ABA will be administered in a form that is acceptable, tolerable, and effective for the subject. Numerous pharmaceutical forms and formulations for biologically active agents are known in the art, and any and all of these are contemplated by the present invention. Thus, for example, the ABA can be formulated in an oral solution, a caplet, a capsule, an injectable, an infusible, a suppository, a lozenge, a tablet, a cream or salve, an inhalant, and the like.

In view of the above methods, it should be evident that the present invention provides ABA for use in contacting cells, such as in treating cells of a subject. The above discussion focuses on the use of ABA alone or as part of a composition for use in what could generally be considered a pharmaceutical or medical setting. However, it is to be understood that the ABA or compositions comprising ABA can be used in treatment of subjects by way of nutritional supplements, such as through dietary supplements. In such supplements, the ABA can be present in doses that are suitable for daily (or more often) administration. Typically, for dietary supplements, the ABA is presented in a form suitable for oral ingestion, such as by way of pill, capsule, tablet, caplet, powder, liquid, or the like. As with the forms for use in medical settings, typical additives can be included, such as colorants, flavorants, binders, gums, and the like.

For example, the ABA may be present as a functional food ingredient, either as a stand-alone ingredient (such as would be seen with sugar, salt, pepper, etc.) or as an ingredient included in the food during processing or packaging. In such situations, the ABA and compositions comprising it would include ABA at an appropriate amount for oral ingestion. It is envisioned that this amount would be considerably less, on a gram of product ingested basis, than the amount used for pharmaceutical use in treating the aforementioned diseases and conditions.

In yet another form of the composition, a nutritional supplement is provided for the treatment of diseases. In this form, the ABA is provided at a higher concentration than the food supplement form, but still at a lower dose than the “pharmaceutical” dose. This form can be considered a supplement for periodic administration of ABA to a subject. For example, it can be likened to a vitamin pill for periodic supplementation of various vitamins and minerals for a person, but rather than providing vitamins and minerals, it would provide ABA to enable the various beneficial effects discussed in this document, as well as others. The dietary supplement may comprise, in addition to ABA, any number of other substances, which are well known as suitable for ingestion by animals and humans. For example, it may contain fillers, binders, gums, colorants, and the like.

As should be evident, the ABA may be provided in a pharmaceutically acceptable form. Thus, ABA can be provided in a form that is suitable for administration to a subject in need of it. It also may be present as a component of a composition, and in particular, a pharmaceutical composition. The ABA may be provided as a purified or semi-purified substance, or as a part of a simple or complex composition. Where present as part of a composition, the composition as a whole should be biologically tolerable at the amount to be exposed to a living cell. Thus, the composition may comprise toxic or otherwise deleterious substances when in its as-produced state, but be rendered non-toxic at a later date by further treatment or simply by dilution. The pharmaceutical composition may comprise any number of substances in addition to ABA, such as, but not limited to, water, salts, sugars, buffers, biologically active compounds, drugs, etc.

The ABA and compositions of the invention can be provided in any suitable form and container. Thus, in another aspect, the invention provides for a container containing ABA or a composition comprising ABA. In this aspect, the ABA or composition will be provided in the container in an amount that is sufficient for at least one use in a method according to the invention. Thus, it can be provided in an amount and in a form that is sufficient for one or more in vitro treatments of a cell for research purposes. It can also be provided in an amount and form that is sufficient for one or more in vivo treatments of a diabetic or person susceptible to developing diabetes. One of skill in the art can immediately contemplate the various numerous other amounts, forms, and uses for the various in vitro and in vivo applications, and thus all such amounts, forms, and uses need not be detailed here.

The container of the invention can be any container, fabricated in any shape and from any suitable material. It thus can be made from plastic, glass, paper or a paper product, metal, or some other polymeric material. It can be in any shape and size, such as in the shape of a tube, vial, ampoule, packet, pouch, wrapper, can, bottle, and box. Those of skill in the medical, dietary supplement, and food arts will immediately recognize the various other shapes, materials, and sizes that are suitable, and therefore these need not be detailed herein.

In yet an additional aspect, the ABA, compositions, and/or containers, or combinations of these, can be provided in the form of a kit. For example, two or more containers containing a pharmaceutical formulation according to the invention may be provided together in a single package, referred to herein as a kit. Likewise, ABA and some or all reagents and supplies necessary for performing an in vitro assay according to the invention can be provided in a single package or kit. Numerous configurations of supplies and reagents may be included in the kit, in accordance with similar kits for pharmaceutical, dietary supplementation, and/or research that are known in the art.

For oral administration, the effective amount of ABA may be administered in, for example, a solid, semi-solid, liquid, or gas state. Specific examples include tablet, capsule, powder, granule, solution, suspension, syrup, and elixir agents. However, the ABA composition is not limited to these forms.

To formulate the ABA of the present invention into tablets, capsules, powders, granules, solutions, or suspensions, the abscisic acid compound is preferably mixed with a binder, a disintegrating agent and/or a lubricant. If necessary, the resultant composition may be mixed with a diluent, a buffer, an infiltrating agent, a preservative and/or a flavor, using known methods. Examples of the binder include crystalline cellulose, cellulose derivatives, cornstarch, and gelatin. Examples of the disintegrating agent include cornstarch, potato starch, and sodium carboxymethylcellulose. Examples of the lubricant include talc and magnesium stearate. Further, additives, which have been conventionally used, such as lactose and mannitol, may also be used.

For parenteral administration, the ABA composition of the present invention may be administered rectally or by injection. For rectal administration, a suppository may be used. The suppository may be prepared by mixing the ABA with a pharmaceutically suitable excipient that melts at body temperature but remains solid at room temperature. Examples include but are not limited to cacao butter, carbon wax, and polyethylene glycol. The resulting composition may be molded into any desired form using methods known to the field.

For administration by injection, the ABA may be injected hypodermically, intracutaneously, intravenously, or intramuscularly. Medicinal drugs for such injection may be prepared by dissolving, suspending or emulsifying the ABA of the invention into an aqueous or non-aqueous solvent such as vegetable oil, glyceride of synthetic resin acid, ester of higher fatty acid, or propylene glycol by a known method. If desired, additives such as a solubilizing agent, an osmoregulating agent, an emulsifier, a stabilizer, or a preservative, which has been conventionally used may also be added. While not required, it is preferred that the composition be sterile or sterilized.

For formulating the ABA of the present invention into suspensions, syrups or elixirs, a pharmaceutically suitable solvent may be used. Included among these is the non-limiting example of water.

The ABA composition of the present invention may also be used together with an additional compound having other pharmaceutically suitable activity to prepare a medicinal drug. A drug, either containing ABA as a stand-alone compound or as part of a composition, may be used in the treatment of subjects in need thereof. In an embodiment, the compound of the present invention can contain ABA and an active pharmaceutical ingredient (API) for reducing hypertension or treating atherosclerosis. APIs for treating hypertension include, but are not limited to, diuretics (such as hydrochlorothiazide, chlorthalidone, chlorothiazide, indapamide, methyclothiazide, metolazone, amiloride, eplerenone, spironolactone, and triamterene), β-blockers (such as acebutolol, atenolol, betaxolol, bisoprolol, carteolol, carvedilol, labetalol, metoprolol, nadolol, penbutolol, pindolol, propranolol, and timolol), calcium channel blockers (such as diltiazem, verapamil, amlodipine, felodipine, isradipine, nicardipine, nifedipine, and nisoldipine), ACE inhibitors (such as benazepril, captopril, enalapril, eosinopril, lisinopril, moexipril, quinapril, ramipril, and trandolapril), and angiotensin II receptor blockers (such as candesartan, eprosartan, irbesartan, losartan, olmesartan, telmisartan, and valsartan). APIs for treating atherosclerosis include, but are not limited to, antiplatelet drugs (such as aspirin, clopidogrel, and ticlopidine), ACE inhibitors, angiotensin II receptor blockers, statins (such as atorvastatin, cerivastatin, fluvastatin, lovastatin, mevastatin, pitavastatin, pravastatin, rosuvastatin, and simvastatin), and thiazolidinediones (such as rosiglitazone, pioglitazone, and troglitazone).

The ABA may also be administered in the form of an aerosol or inhalant prepared by charging the abscisic acid in the form of a liquid or fine powder, together with a gaseous or liquid spraying agent and, if necessary, a known auxiliary agent such as an inflating agent, into a non-pressurized container such as an aerosol container or a nebulizer. A pressurized gas of, for example, dichlorofluoromethane, propane or nitrogen may be used as the spraying agent.

ABA may be administered to an animal, preferably mammals such as humans, in need thereof as a pharmaceutical or veterinary composition, such as tablets, capsules, solutions, or emulsions. In a preferred embodiment, the free acid form of punicic acid is administered. However, administration of other forms of ABA, including but not limited to esters thereof, pharmaceutically-suitable salts thereof, metabolites thereof, structurally related compounds thereof, analogs thereof, and combinations thereof, in a single dose or a multiple dose, are also contemplated by the present invention.

Abscisic acid may also be administered to an animal in need thereof as a nutritional additive, either as a food or nutraceutical supplement.

The ABA is preferably used and/or administered in the form of a composition. Suitable compositions are, preferably, a pharmaceutical composition, a foodstuff or a food supplement. These compositions provide a convenient form in which to deliver the ABA. Compositions of the invention may comprise an antioxidant in an amount effective to increase the stability of the ABA with respect to oxidation.

The amount of ABA that is administered in the method of the invention or that is for administration in the use of the invention is any suitable amount sufficient to be effective. It is preferably about 1-3000 mg/day, more preferably about 10-2000 mg/day, most preferably about 500-1000 mg/day for an 80 kg subject. Suitable compositions can be formulated accordingly. Those of skill in the art of dosing of biologically active agents will be able to develop particular dosing regimens for various subjects based on known and well understood parameters.

A preferred composition according to the invention is a foodstuff. Food products (which term includes animal feed) preferably contain a fat phase, wherein the fat phase contains the ABA. The foodstuffs are optionally used as a blend with a complementary fat. For example, the fat may be selected from: cocoa butter, cocoa butter equivalents, palm oil or fractions thereof, palmkernel oil or fractions thereof, interesterified mixtures of those fats or fractions thereof. It may also contain liquid oils, such as those selected from: sunflower oil, high oleic sunflower oil, soybean oil, rapeseed oil, cottonseed oil, fish oil, safflower oil, high oleic safflower oil, corn oil, and MCT-oils. Examples of suitable foodstuffs include those selected from the group consisting of margarines, fat continuous or water continuous or bicontinuous spreads, fat reduced spreads, confectionery products such as chocolate or chocolate coatings or chocolate fillings or bakery fillings, ice creams, ice cream coatings, ice cream inclusions, dressings, mayonnaises, cheeses, cream alternatives, dry soups, drinks, cereal bars, sauces, snack bars, dairy products, clinical nutrition products, and infant formulations.

Other non-limiting examples of compositions are pharmaceutical compositions, such as in the form of tablets, pills, capsules, caplets, multiparticulates (including granules, beads, pellets and micro-encapsulated particles); powders, elixirs, syrups, suspensions, and solutions. Pharmaceutical compositions will typically comprise a pharmaceutically acceptable diluent or carrier. Pharmaceutical compositions are preferably adapted for administration parenterally (e.g., orally). Orally administrable compositions may be in solid or liquid form and may take the form of tablets, powders, suspensions, and syrups, among other things. Optionally, the compositions comprise one or more flavoring and/or coloring agents. In general, therapeutic and nutritional compositions may comprise any substance that does not significantly interfere with the action of the ABA on the subject.

Pharmaceutically acceptable carriers suitable for use in such compositions are well known in the art of pharmacy. The compositions of the invention may contain 0.01-99% by weight of ABA. The compositions of the invention are generally prepared in unit dosage form. Preferably, the unit dosage of ABA is about 1-3000 mg, more preferably about 10-1000 mg. The excipients and/or carriers used in the preparation of these compositions are known in the art.

Further examples of product forms for the composition are food supplements, such as in the form of a soft gel or a hard capsule comprising an encapsulating material selected from the group consisting of gelatin, starch, modified starch, starch derivatives such as glucose, sucrose, lactose, and fructose. The encapsulating material may optionally contain cross-linking or polymerizing agents, stabilizers, antioxidants, light absorbing agents for protecting light-sensitive fills, preservatives, and the like. Preferably, the unit dosage of ABA in the food supplements is about 1-3000 mg, more preferably about 10-1000 mg.

In general, the term carrier may be used throughout this application to represent a composition with which ABA may be mixed, be it a pharmaceutical carrier, foodstuff, nutritional supplement or dietary aid. The materials described above may be considered carriers of ABA for the purposes of the invention. Preferably, the carrier has little to no biological activity.

The method of the present invention can comprise administering a therapeutically effective amount of ABA to an animal in need thereof. The effective amount of ABA depends on the form of the ABA compound administered, the duration of the administration, the route of administration (e.g., oral or parenteral), the age of the animal and the condition of the animal, including mammals and humans.

For instance, an amount of ABA effective to treat or prevent hypertension or vascular inflammation can range from about 1 to about 8000 mg/day for an 80 kg subject. A preferred effective amount of ABA is about 50-3000 mg/day, with a more preferred dose being about 100-2000 mg/day for an 80 kg subject. The upper limit of the effective amount to be administered is not critical, as ABA is relatively non-toxic as long as the recipient's diet contains the necessary essential nutrients. The ABA is most effective in treating or preventing hypertension or vascular inflammation of an subject when administered for periods ranging from about 1 to about 360 days, preferably 1 to 120 days, and a more preferably 1 to 60 days.

Those of ordinary skill in the art will readily optimize effective dosages and administration regimens as determined by good medical practice and the clinical condition of the individual patient.

The frequency of dosing will depend on the pharmacokinetic parameters of the compounds and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose is calculated according to body weight, body surface areas or organ size. The availability of animal models is particularly useful in facilitating a determination of appropriate dosages of a given therapeutic. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Typically, appropriate dosages are ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions. Those studies, however, are routine and within the level of skilled persons in the art.

It will be appreciated that the compositions and treatment methods of the invention are useful in fields of human medicine and veterinary medicine. Thus, the subject to be treated is a mammal, such as a human or other mammalian animal. For veterinary purposes, subjects include for example, farm animals including cows, sheep, pigs, horses and goats, companion animals such as dogs and cats, exotic and/or zoo animals, and laboratory animals including mice, rats, rabbits, guinea pigs and hamsters.

Without further description, it is believed that one of ordinary skill in the art can, using the preceding description and the following illustrative examples, make and utilize the present invention. The following example is given to illustrate the present invention. It should be understood that the invention is not to be limited to the specific conditions or details described in the example.

### Example

### Materials and Methods

Animal Procedures

Five-week old ApoE −/− mice (n=40) were housed at the animal facilities at Virginia Polytechnic Institute and State University in a room maintained at 75° F., with a 12:12 h light-dark cycle starting from 6:00 AM. All experimental procedures were approved by the Institutional Animal Care and Use Committee of Virginia Polytechnic Institute and State University and met or exceeded requirements of the Public Health Service/National Institutes of Health and the Animal Welfare Act. Mice were fed a high saturated fat (AIN-93G-based) diet containing 19.6% fat and 0.2% total cholesterol and obtaining 40.1% of Kcal from fat (4.4 Kcal/g) with or without 100 mg/kg of a racemic ABA mixture (Guri et al., J. Nutr. Biochem. 19:216-28, 2008). Systolic blood pressure was assessed on days 0, 28, 56, and 72. On day 84, fasted mice (12 h) were sacrificed by carbon dioxide asphyxiation and blood was withdrawn through cardiac puncture. Glucose concentrations of whole blood were assessed with an Accu-Chek® Glucometer (Roche, Indianapolis, Ind.) and plasma insulin was measured through insulin ELISA (Millipore, Billerica, Mass.). Aortas were collected for analysis of immune cell infiltration, histological evaluation of atherosclerotic lesions, and gene expression analyses.

Blood Pressure Assessment

Systolic blood pressure was measured with a computerized non-invasive tail-cuff system (CODA 2 mouse rat blood pressure system; Kent Scientific Corporation, Torrington, Conn.). Conscious animals were kept in a restrainer, with a standard acclimatization time of 10 min and gentle heating of the tail before every recording session. Fifteen recordings, which were preceded by five acclimitization recordings, were performed on each animal during each timepoint.

Plasma Lipid and FPLC Analysis

Plasma cholesterol and triglyceride levels were measured using kits from Raichem (San Diego, Calif.) adapted for microtiter plate assays. Plasma free fatty acids were measured using the NEFA-HR(2) kit from Wako. Fast performance liquid chromatography (FPLC) separation of plasma lipoproteins was performed on a Superose 6 column using buffer containing 0.15M NaCl, 0.01M Na₂HPO₄ and 1 mM EDTA at a flow rate of 0.5 ml/min. Forty 0.5 ml fractions were collected and cholesterol was measured in fractions 10-40. Fractions 15-20 contain VLDL, fractions 21-27 contain LDL and fractions 28-34 contain HDL.

Digestion of Aorta

After euthanization, mouse hearts were perfused with PBS for 2 minutes. Aortas were then extracted. Aortic roots were cut into small >1 mm³ pieces, and placed in 1× Hanks Balanced Sodium Salt Solution (HBSS) containing 0.2% type II collagenase (Worthington Biochemicals). After digestion at 37° C. for 1 hour, samples were sent through a 100 mm nylon filter (BD), washed with HBSS, and centrifuged at 1200 rpm for 10 min. Cell suspensions were then resuspended in FACS buffer and enumerated by using a Z1 Single Particle Counter (Beckman Coulter).

Flow Cytometry

Aortic root wall-derived cells (i.e, tunica intima, media and adventitia) were seeded into 96-well plates and centrifuged at 4° C. at 3000 rpm for 4 minutes. To assess differential monocyte/macrophage infiltration, the cells were then incubated in the dark at 4° C. for 20 minutes in FcBlock (20 μg/ml; BD Pharmingen) for macrophage assessment, and then for an additional 20 minutes with fluorochrome-conjugated primary antibodies anti-F4/80-PE-Cy5 (5 μg/mL, ebioscience) and anti-CD11b-FITC (2 μg/mL, eBioscience). For lymphocyte assessment, cells were incubated with CD4-FITC (2 μg/mL; BD Pharmingen) and CD3 PE-Cy5 (2 μg/mL; BD Pharmingen). After incubation with primary antibodies, cells were centrifuged at 4° C. at 3000 rpm for 4 minutes and washed with 200 μl of FACS buffer. After washing, cells were suspended in 200 μL PBS and 3-color data acquisition was performed on a FACS Calibur flow cytometer. Data analyses were performed by using the CellQuest software (BD).

Histology Analyses of the Aorta

The aortic root sections used for histological analyses were perfusion-fixed with paraformaldehyde. Samples of aortas were cut transversally, stained with hematoxylin and eosin, and analyzed histologically. Areas for the lumen, tunica media, and aortic lesions and the thicknesses of the tunica intima and media were assessed using Image Pro Plus 6.1 software (Media Cybernetics, Bethesda, Md.). For thickness analyses, eight different measurements from the intima and media were taken using previously described methods (Hazell et al., Free Radic. Biol. Med. 31:1254-62, 2001). For semiquantitative assessment of lesion severity, methods described by van Vlijmen et al were used (van Vlijmen et al., J. Clin. Invest. 93:1403-10, 1994). Briefly, lesions were classified into five categories (1) early fatty streak: per section up to 10 foam cells present in the intima, (2) regular fatty streak: more than 10 cells present in the intima, (3) mild plaque: extension of foam cells into the media and mild fibrosis of the media without significant loss in architecture, (4) moderate plaque: foam cells in media, fibrosis, cholesterol clefts, mineralization, and/or necrosis of the media, (5) severe plaque: as 4 but more extensive and deeper in the media. Scoring classifications were applied to 15 lesions from control and ABA-fed mice. Lesions falling in categories 1-3 were classified as mild and 4-5 as severe.

Quantitative Real-Time Reverse Transcriptase PCR

Total RNA was isolated from aortas using the RNA isolation Minikit (Qiagen) according to the manufacturer's instructions. Total RNA (1 μg) was used to generate a complementary DNA (cDNA) template using the iScript cDNA Synthesis Kit (Bio-Rad, Hercules, Calif.). The total reaction volume was 20 μL with the reaction incubated as follows in an MJ MiniCycler: 5 minutes at 25° C., 30 minutes at 52°, 5 minutes at 85° C., and hold at 4° C. PCR was performed on the cDNA using Taq DNA polymerase (Invitrogen, Carlsbad, Calif.) and using previously described conditions (Bassaganya-Riera et al., Gastroenterology 127:777-91, 2004). Each gene amplicon was purified with the MiniElute PCR Purification Kit (Qiagen) and quantitated on an agarose gel by using a DNA mass ladder (Promega). Those purified amplicons were used to optimize real-time PCR conditions and to generate standard curves in the real-time PCR assay. Primer concentrations and annealing temperatures were optimized for the iCycler iQ system (Bio-Rad) for each set of primers using the system's gradient protocol. PCR efficiencies were maintained between 92 and 105% and correlation coefficients above 0.98 for each primer set during optimization and also during the real-time PCR of sample DNA.

Complementary DNA (cDNA) concentrations for genes of interest were examined by real-time quantitative PCR using an iCycler IQ System and the iQ SYBR green supermix (Bio-Rad). A standard curve was generated for each gene using 10-fold dilutions of purified amplicons starting at 5 pg of cDNA and used later to calculate the starting amount of target cDNA in the unknown samples. SYBR green I is a general double-stranded DNA intercalating dye and may therefore detect nonspecific products and primer/dimers in addition to the amplicon of interest. In order to determine the number of products synthesized during the real-time PCR, a melting curve analysis was performed on each product. Real-time PCR was used to measure the starting amount of nucleic acid of each unknown sample of cDNA on the same 96-well plate. Results are presented as a starting quantity of target cDNA (picograms) per microgram of total RNA. Primer sequences, the length of the PCR product, and gene accession numbers are outlined in Table 1.

TABLE 1
Oligonucleotide sequences for quantitative real-time PCR.^{a,b}

| | | Length | Accession |
|---|---|---|---|
| Primer | Sequence | (bp) | Number |
| β-ActinF | 5′-CCCAGGCATTGCTGACAGG-3′ | 141 | X03672 |
| | (SEQ ID NO: 1) | | |
| β-ActinR | 5′-TGGAAGGTGGACAGTGAGGC-3′ | | |
| | (SEQ ID NO: 2) | | |
| eNOSF | 5′-CCCGGGACTTCATCAATCAGT-3′ | 121 | NM_008713 |
| | (SEQ ID NO: 3) | | |
| eNOSR | 5′-TCCCGGAGCTGGTAGGTG-3′ | | |
| | (SEQ ID NO: 4) | | |
| E-selectinF | 5′-CAGCTTTGCATGATGGCGTCT-3′ | 83 | NM_011345 |
| | (SEQ ID NO: 5) | | |
| E-selectinR | 5′-GAAGGGTACAGGCGAGTTGG-3′ | | |
| | (SEQ ID NO: 6) | | |
| 15-LOXF | 5′-GCGCAGCACTCTTCCATCC-3′ | 106 | NM_009660 |
| | (SEQ ID NO: 7) | | |
| 15-LOXR | 5′-TCGTCGCGTCCTTGGTTTTA-3′ | | |
| | (SEQ ID NO: 8) | | |
| MCP-1F | 5′-CTTGCCTAATCCACAGACTG-3′ | 146 | AJ238892 |
| | (SEQ ID NO: 9) | | |
| MCP-1R | 5′-GCCTGAACAGCACCACTA-3′ | | |
| | (SEQ ID NO: 10) | | |
| VCAM-1F | 5′-TCTCCCAGGAATACAACGAT-3′ | 75 | NM_011693 |
| | (SEQ ID NO: 11) | | |
| VCAM-1R | 5′-ACAGGTCATTGTCACAGCAC-3′ | | |
| | (SEQ ID NO: 12) | | |
| PPAR γF | 5′CAGGCTTGCTGAACGTGAAG3′ | 117 | NM_011146 |
| | (SEQ ID NO: 13) | | |
| PPAR γR | 5′GGAGCACCTTGGCGAACA3′ | | |
| | (SEQ ID NO: 14) | | |
| CD36F | 5′CCGGGCCACGTAGAAAACA3′ | 156 | NM_007643 |
| | (SEQ ID NO: 15) | | |
| CD36R | 5′CCTCCAAACACAGCCAGGAC3′ | | |
| | (SEQ ID NO: 16) | | |

^{a}F = forward; R = reverse. PCR primer pairs were designed for an optimal annealing temperature of 57° C. and product lengths between 75 and 150 base pairs.
^{b}When plotting threshold cycle versus log starting quantity (pg), standard curves had slopes between −3.1 and −3.7; PCR efficiencies between 92 and 105 and R² above 0.98.

Determination of MCP-1 Concentrations in Plasma

A Ready-set-go MCP-1 ELISA (ebioscience) was used to quantify plasma MCP-1 according to the manufacturer's instructions.

Intracellular cAMP and NO Concentrations and eNOS mRNA in Human Aorta Endothelial Cells

Confluent human aorta endothelial cells (HAECs) at passage 3 maintained with EGM-2 media (Clonetics, Walkerville, Md.) were adapted to Hank's buffered salt solution buffer for 30 min and then stimulated with ABA or forskolin (1 μM) for 10 min. For intracellular assessment of cAMP, the cells were harvested into 0.1 M HCL and the lysates were obtained by sonication and centrifugation. cAMP the lysates were determined by using an EIA kit (Assay Designs, Ann Arbor, Mich.) and results were obtained from four independent experiments. Nitric oxide levels in the cell culture supernatants were determined with the Nitric Oxide Assay Kit according to manufacturer's instructions (Assay Designs). We also assessed whether treatment of HAECs with ABA would enhance eNOS mRNA expression. HAECs were treated with or without 10 μM ABA for 24 hours and eNOS mRNA expression was measured by real time PCR.

Statistics

Data from day 84 were analyzed as a completely randomized design. Blood pressure data were analyzed using repeated measures. To determine the statistical significance of the model, analysis of variance (ANOVA) was performed using the general linear model procedure of Statistical Analysis Software (SAS), and probability value (P)<0.05 was considered to be significant.

Results

ABA Supplementation has No Effect on Body Weight, Cholesterol, Glucose or Insulin Concentrations but Increases Triglycerides and Non-Esterified Fatty Acids

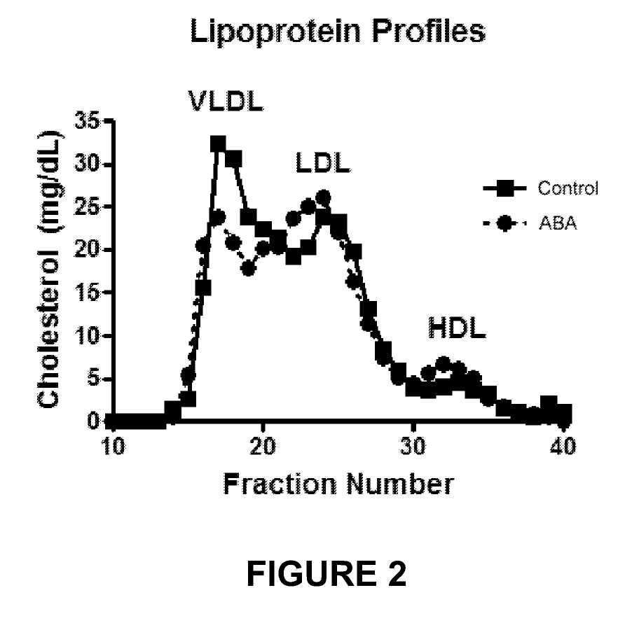

ApoE-deficient mice were fed high-fat diets with or without ABA for 84 days. Dietary ABA-supplementation had no significant effect on body weights, plasma glucose, insulin, or total cholesterol levels (Table 2). ABA did significantly raise plasma triglyceride (TG) and non-esterified fatty acid concentrations. In our FPLC analysis, we observed a slight shift in the profiles to lower VLDL and higher LDL and HDL in the ABA-fed mice, indicative of a more triglyceride-rich VLDL (FIG. 2).

TABLE 2
Effect of ABA supplementation on metabolic parameters^{a,b}

Diet

| | Control | ABA |
|---|---|---|
| Body Weight (g) | 29.7 ± 0.63 | 30.8 ± 0.63 |
| Glucose (mg/dL) | 195.1 ± 11.9 | 194.6 ± 11.9 |
| Insulin (ng/mL) | 0.413 ± 0.05 | 0.432 ± 0.05 |
| HOMA-IR | 4.85 ± 0.76 | 4.93 ± 0.76 |
| Cholesterol (mg/dL) | 318.1 ± 23.8 | 339.0 ± 25.1 |
| TGs (mg/dL) | 90.5 ± 12.3 | 136.1 ± 12.3* |
| NEFAs (mequiv./mL) | 1.51 ± 0.17 | 2.05 ± 0.17* |

^{a}ApoE-deficient mice were fed high-fat diets with or without abscisic acid (ABA, 100 mg/kg) for 84 days. Plasma from fasted mice (12 h) on day 84 were analyzed for metabolic parameters.
^{b}Data are presented as least square means ± standard error. Data points with an asterisk (*) are significantly different (P < 0.05).
HOMA-IR = homeostasis model assessment-insulin resistance;
TGs = triglycerides;
NEFAs = non-esterified (“free” or unsaturated) fatty acids.

Dietary ABA Supplementation Improves Systolic Blood Pressure

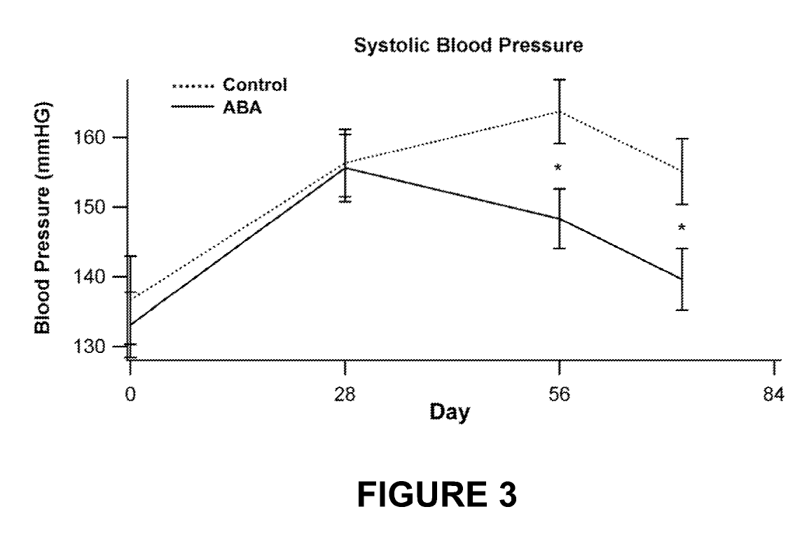

To determine whether ABA affects hypertension in ApoE-deficient mice we assessed blood pressure on days 0, 28, 56, and 72 of the dietary intervention. On days 56 and 72 mice fed ABA had significantly reduced systolic blood pressure when compared to control-fed mice (FIG. 3). ABA had no significant affect on diastolic blood pressure or mean heart rate (data not shown).

ABA Decreases Aortic Root Wall Thickness, Elevates Aortic eNOS and 15-LOX mRNA Expression, and Reduces Atherosclerotic Lesions

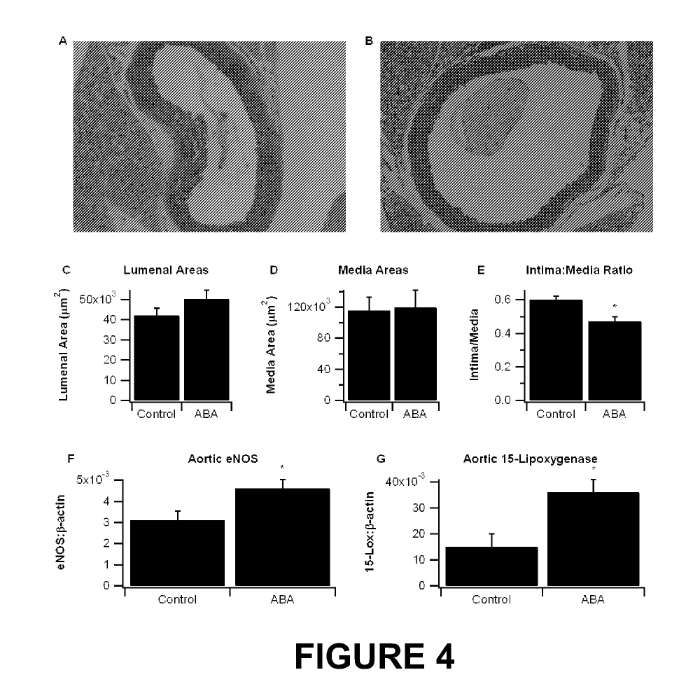

In line with the decrease in systolic blood pressure, there were significant histological differences in the aortas of control and ABA-fed mice (FIG. 4). While there were no significant differences in luminal and media areas, aortas from ABA-supplemented mice had a significantly lower intima to media ratio (FIG. 4C-3E). These differences were mainly attributable to reductions in intimal wall thickness. ABA also significantly elevated aortic expression of eNOS and 15-lipoxygenase (FIGS. 4F and 3G), two genes involved in vasorelaxation.

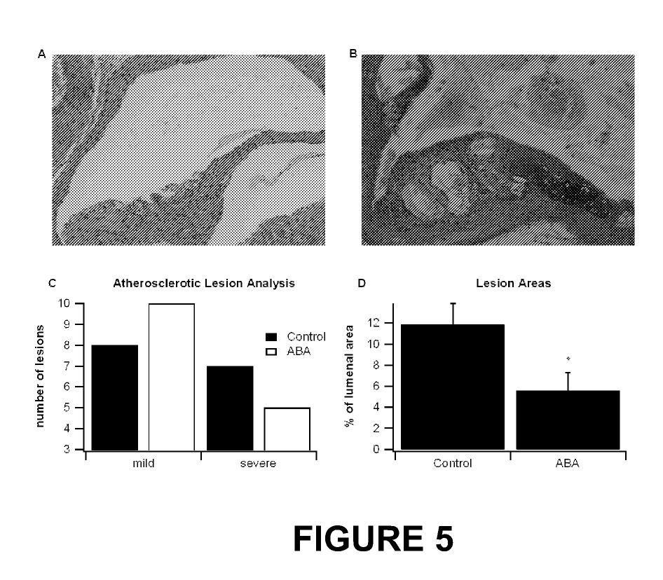

To assess the affect of ABA-supplementation on atherosclerotic lesions, a semiquantitative analysis of lesion severity was performed. Fifteen lesions from control and ABA-fed mice were analyzed histologically and classified as either mild or severe using criteria described by van Vlijmen et al. Eight of the 15 lesions in control-fed mice were severe, whereas in the ABA-fed mice 10 were mild and 5 were severe (FIG. 5A-4C). Because atherosclerotic plaque is a significant contributor to vascular thrombosis and stenosis, we also assessed lesion areas as a percent of lumenal area, as described by Andersson et al. (Atherosclerosis 188:331-40, 2006). We found that mice fed ABA had a significantly decreased lesion area as a percent of total lumenal area (FIG. 5D).

ABA Suppresses Aortic Inflammation

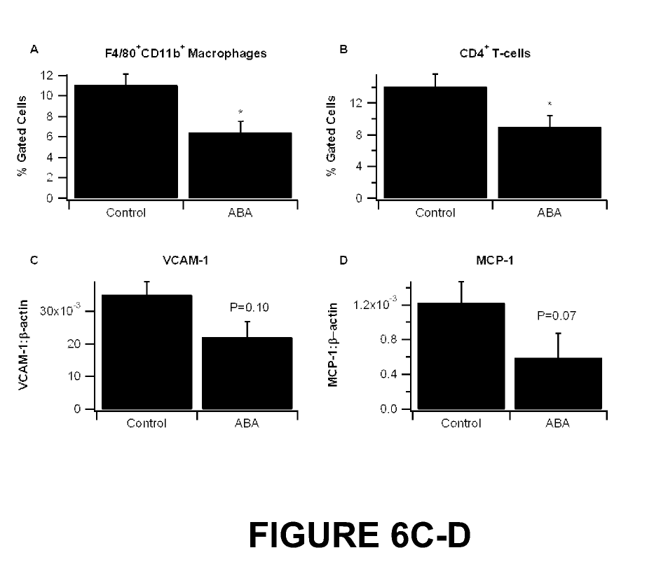

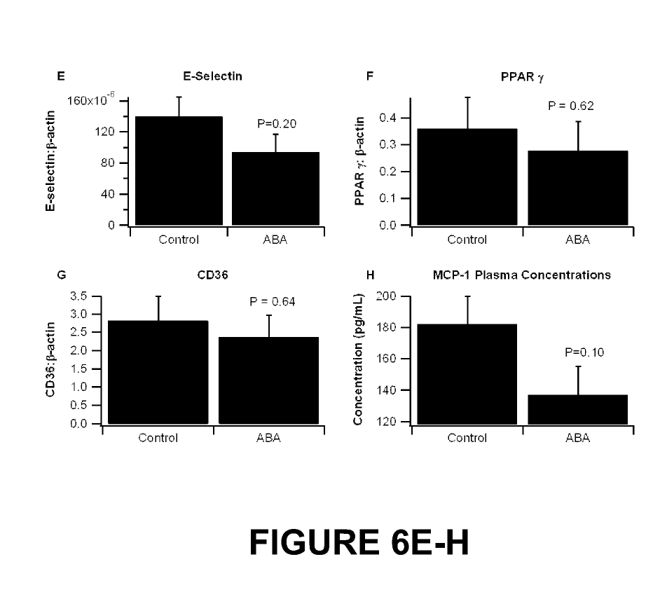

Atherosclerosis is associated with an infiltration of macrophages and lymphocytes into the atherosclerotic plaque and a vascular remodeling of aortic wall which leads to stenosis (Shimokama et al., Pathol. Int. 45:801-814, 1995; and Lutgens et al., Arterioscler. Thromb. Vasc. Biol. 21:1359-65, 2001). Our previous findings have shown that ABA-supplementation significantly reduces macrophage infiltration into white adipose tissue and induces an approximately 15-fold reduction in MCP-1 expression in the adipose tissue stromal-vascular fraction of obese db/db mice (Guri et al., 2008). These effects were dependent on the presence of PPAR γ in immune cells. We demonstrate here that ABA-supplementation significantly reduces recruitment of two specific immune cell populations, F4/80⁺CD11b⁺ macrophages and CD4⁺ T-cells, in the aortic root (FIG. 6A). The mRNA expression of pro-inflammatory markers VCAM-1 (P=0.07), MCP-1 (P=0.10), and E-selectin (P=0.20) were reduced, though not to statistical significance, in the aortic roots of ABA-fed mice, and plasma MCP-1 concentrations were decreased (P=0.10, FIG. 6B-6H). There were no differences in the expression of PPAR γ or its responsive genes in the aorta

ABA Increases Intracellular Concentration of Intracellular cAMP and Stimulates NO Production in Aortic Endothelial Cells

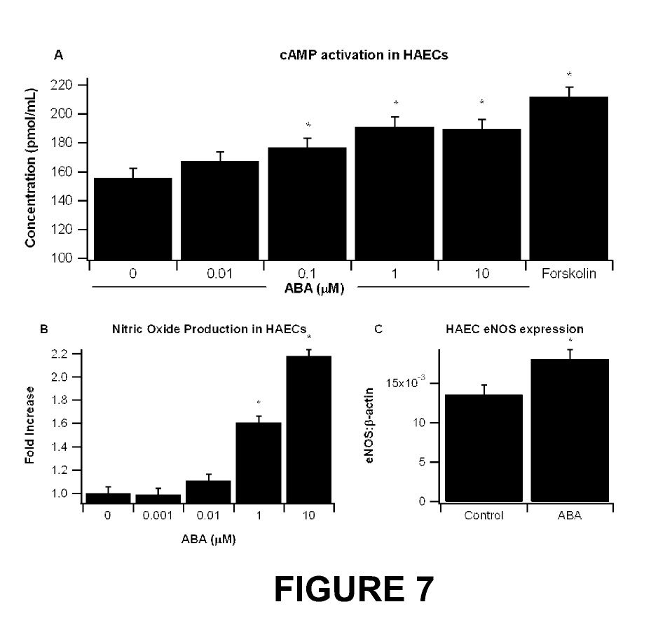

Other reports demonstrate that ABA increases intracellular levels of cAMP in pancreatic β-cells (Bruzzone et al., J. Biol. Chem. 283:32188-97, 2008). Our findings in the aorta showing increased expression of eNOS and a downregulated trend in pro-inflammatory markers VCAM-1 and MCP-1 could be due to changes in this second messenger. To determine whether ABA increases cAMP in endothelial cells, we treated HAECs with ABA or the cAMP activator forskalin. ABA significantly increased intracellular cAMP concentrations at doses as low as 0.1 μM ABA, with 1 μM ABA inducing maximal effect (FIG. 7A). Increased cAMP concentrations correlated with enhanced NO production in ABA-treated endothelial cells (FIG. 7B). HAECs were treated with or without 10 μM ABA for 24 hours, total RNA was isolated and eNOS mRNA expression was measured by real time PCR. We found that ABA significantly (P<0.02) increased eNOS mRNA levels (FIG. 7C).

### DISCUSSION

CVD is the primary leading cause of death in the United States (Rosamond et al., Circulation 117:e25-146, 2008). CVD is associated with atherosclerosis, the progressive build-up of plaque in the arterial walls, which over time can lead to occlusion of blood vessels. The objective of this study was to determine the effects of ABA on the development of atherosclerosis. To address that goal, ApoE-deficient mice were fed either a control or ABA-supplemented high-fat, atherogenic diet for 84 days. The progression of atherosclerosis and lesion development in ApoE-deficient mice occurs in a similar manner to that of humans. Also similar to humans, ApoE-deficient mice undergo accelerated atherosclerosis when fed high-fat diets, with histological differences being observed as early as six weeks of age (Nakashima et al., Arterioscler. Thromb. 14:133-40, 1994).

We first found differences between the groups on day 56, where we observed a significant decrease in mean systolic blood pressure in ABA-fed mice. That trend also continued at day 72, in which the differences were somewhat accentuated. Those in vivo findings are in line with the increased expression of eNOS and 15-LOX in aortas of ABA-fed mice and increased synthesis of nitric oxide in ABA-treated endothelial cells, though more corroborative studies are needed to validate the importance of nitric oxide and 15-LOX-metabolites to ABA's antihypertensive effects. Interestingly, 15-LOX catalyzes the generation of arachidonic acid and linoleic acid-derived lipid mediators (e.g., 15-HETE and 13-HODE) which elicit anti-inflammatory and cardioprotective effects (Wittwer et al., Prostaglandins Leukot. Essent. Fatty Acids 77:67-77, 2007). There are also studies showing that 15-LOX can exert a pro-inflammatory, atherogenic effect through increasing LDL oxidation, smooth muscle proliferation, and monocyte recruitment into the vessel wall (Wittwer et al., 2007). We did not observe increased aortic inflammation with the upregulation of 15-LOX. In fact, aortic root walls from ABA-fed mice were less inflamed than those from control fed-mice. Dietary ABA-supplementation significantly reduced the percent of F4/80⁺CD11b⁺ macrophages and CD4⁺ lymphocytes, which are known to contribute to atherosclerotic plaque formation and obesity-related inflammation (Bassaganya-Riera et al., Cell Immunol. 258:138-46, 2009). Also, mRNA for MCP-1, VCAM-1, and E-selectin and MCP-1 plasma protein levels tended to be decreased. In line with reduced inflammation, ABA-supplemented mice had fewer and less severe aortic lesions, and the area of aortic lesions was significantly reduced.

Based on a recent report by Bruzzone et al that ABA increases cAMP in pancreatic islets (Bruzzone et al., J. Biol. Chem. 283:32188-97, 2008), we assessed whether ABA induced a similar effect in endothelial cells, finding a dose-dependent relationship between ABA levels and cAMP concentration. cAMP signaling plays a major role in maintaining vascular homeostasis by inhibiting endothelial (D'Angelo et al., J. Cell. Biochem. 67:353-66, 1997) and smooth muscle cell (Indolfi et al., Nat. Med. 3:775-9, 1997) proliferation, decreasing immune cell endothelial adhesion (Morandini et al., Am. J. Physiol. 270:H807-16, 1996) and maintaining endothelial barrier function (Moy et al., Am. J. Physiol. 274:L1024-9, 1998; and Lum et al., Am. J. Physiol. 277:C580-8, 1999). The expression eNOS, which we show is increased by ABA-treatment in vivo and in vitro, is also regulated in part through the CRE-responsive elements in its promoter region (Michell et al., J. Biol. Chem. 276:17625-8, 2001; and Niwano et al., Circ. Res. 93:523-30, 2003). A recent study has shown that the lanthione synthetase C-like protein (LANCL2) is required for ABA binding to the membrane of human granulocytes and ABA signaling (Sturla et al., J. Biol. Chem. 284: 28045-57, 2009). Additional loss-of-function studies are needed to determine whether ABA's effect on cAMP is LANCL2-dependent.

In our previous studies with db/db mice, ABA-supplementation improved glucose tolerance and adipose tissue inflammation through a mechanism that was partially dependent on PPAR γ (Guri et al., Clin. Nutr. 26:107-16, 2007; and Guri et al., 2008), a transcription factor that inhibits NF-κB activation in macrophages (Pascual et al., Nature 437:759-63, 2005). Here, we report no significant differences in PPAR γ or its responsive genes in the aorta, a finding which of itself does not rule out ABA from still activating PPAR γ in immune cells. In this case, the RNA from macrophages and T cells infiltrating the tunica intima of the aortic root may have been diluted by RNA originated from endothelial, smooth muscle cells and fibroblasts (i.e., intima, media and adventitia).

Recently, Magnone et al. (J. Biol. Chem. 284: 17808-18, 2009) investigated the effect of ABA on vascular smooth muscle cells and primary human monocytes in vitro, and found that ABA increases MCP-1 and MMP-9 when stimulated in culture for 6 hours through Ca²⁺/protein kinase C induced phosphorylation of NF-κB. The authors concluded that ABA is pro-atherogenic, an interpretation which differs significantly from our in vivo findings. The report by Magnone et al demonstrates the presence of endogenous ABA in human arterial atherosclerotic plaques at a ten-fold greater concentration in comparison to uninvolved tissue. While the authors of that report suggest that the presence of ABA in the plaques contributes to the pathogenesis of atherosclerosis, in light of our in vivo findings demonstrating improved intima to media ratio and decreased atherosclerotic lesions in ABA-fed mice, we suggest that the body synthesizes ABA as a part of a down-regulatory mechanism designed to dampen lesion development during atherosclerosis.

In mouse models of overweight, obesity, and diabetes we have consistently observed that ABA supplementation ameliorates glucose tolerance, adipose tissue inflammation, and liver steatosis (Guri et al., 2007; and Guri et al., 2008). In the present study we found that ABA decreases the expression of inflammatory genes in the aorta and alleviates hypertension. This apparent paradoxical role of ABA, to increase inflammation in some conditions and lessen it in others, would be in line with ABA's role as an endogenous or dietary modulator of stress response, which is one of its many roles in plants. NEFAs, which we found were increased in the plasma of ABA-fed mice, have also been described as endogenous homeostatic regulators of stress and protect endothelial cells from the cytotoxic effects of hydrogen peroxide (Karman et al., J. Lab. Clin. Med. 129:548-56, 1997). However, the increase in plasma TG caused by ABA in this study is opposite to what we anticipated and to what our metabolic studies in db/db mice showed (Guri et al., 2008). Our finding that ABA improves atherosclerosis despite increasing plasma TG levels and does not affect total cholesterol levels may be linked to its antihypertensive effects, as there is considerable epidemiological evidence suggesting that blood pressure effects atherosclerotic lesion development (Chobanian et al., Arch. Intern. Med. 156:1952-6, 1996). In line with these reports in humans, ApoE-deficient mice lacking eNOS develop more severe atherosclerotic lesions (Knowles et al., J. Clin. Invest. 105:451-8, 2000).

In conclusion, our data indicates that ABA-supplementation elicits a local anti-atherogenic effect in the arterial wall of ApoE-deficient mice and supports its use as a preventive and therapeutic intervention.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A method for treating or preventing hypertension, the method comprising administering to the mammal a composition containing one or more compounds selected from the group consisting of abscisic acid (ABA) in its free acid form, esters thereof, pharmaceutically suitable salts thereof, metabolites thereof, structurally related compounds thereof, and analogs thereof.

2. The method of claim 1, wherein the composition further comprises a carrier.

3. The method of claim 2, wherein the carrier is a pharmaceutical excipient.

4. The method of claim 2, wherein the carrier is a nutritional supplement, functional food, or dietary aid.

5. The method of claim 1, wherein the purity of the one or more compounds is greater than about 95%.

6. The method of claim 1, wherein the one or more compounds is abscisic acid in its free acid form.

7. The method of claim 5, wherein the abscisic acid in its free acid form is chemically synthesized.

8. The method of claim 1, wherein the one or more compound is administered at a dose of about 0.0125 mg/kg/day to about 37.5 mg/kg/day.

9. A method for treating or preventing vascular inflammation, the method comprising administering to the mammal a composition containing one or more compounds selected from abscisic acid (ABA) in its free acid form, esters thereof, pharmaceutically suitable salts thereof, metabolites thereof, structurally related compounds thereof, and analogs thereof.

10. The method of claim 9, wherein the vascular inflammation is atherosclerosis, myocardial infarction, vasculitis or stroke.

11. The method of claim 9, wherein the amount of the one or more compounds is sufficient to decrease infiltration of macrophages and lymphocytes into atherosclerotic plaques or blood vessels with inflammatory lesions.

12. The method of claim 9, wherein the composition further comprises a carrier.

13. The method of claim 12, wherein the carrier is a pharmaceutical excipient.

14. The method of claim 12, wherein the carrier is a nutritional supplement, functional food, or dietary aid.

15. The method of claim 9, wherein the purity of the one or more compounds is greater than about 95%.

16. The method of claim 9, wherein the one or more compounds is abscisic acid in its free acid form.

17. The method of claim 16, wherein the abscisic acid in its free acid form is chemically synthesized.

18. The method of claim 9, wherein the one or more compound is administered at a dose of 0.0125 mg/kg/day to about 37.5 mg/kg/day.

19. A composition comprising
a) one or more compounds selected from the group consisting of abscisic acid (ABA) in its free acid form, esters thereof, pharmaceutically suitable salts thereof, metabolites thereof, structurally related compounds thereof, and analogs thereof; and
b) an active pharmaceutical ingredient for treating hypertension or vascular inflammation.

20. The composition of claim 19, wherein the vascular inflammation is atherosclerosis, myocardial infarction, vasculitis, or stroke.
